(19) United States
(12) Patent Application Publication
(10) Pub. No.: US 2006/0142666 A1
(43) Pub. Date: Jun. 29, 2006

(54) Method of indicating the value of a sampled heartrate

(76) Inventor: Richard D. Ciervo, New Britain, CT (US)

Correspondence Address: Arthur G. Schaier;Carmody & Torrance LLP, 50 Leavenworth Street, P.O. Box 1110, Waterbury, CT, 06721-1110, US

(21) Appl. No.: 10/627,093

(22) Filed: Jul. 25, 2003

(51) Int. Cl. A61B 5/04

(52) U.S. Cl. 600/519

(57) ABSTRACT

A method of generating an out of zone alert when a sampled biomedical value does not satisfy a condition, wherein the generation of the out of zone alert is performed by an indicator, and the sampled biomedical value is determined in a heartrate monitoring system, and further wherein the indicator does not generate the out of zone alert when the sampled biomedical value fails to satisfy the condition until a sampled biomedical value is determined to satisfy the condition, the method comprising the steps of determining when a sampled biomedical value satisfies the condition; and thereafter, permitting the generation of the out of zone alert when the sampled biomedical value is determined not to satisfy the condition.

### FIELD OF THE INVENTION

The present invention relates to heartrate monitors in general, and in particular, to an improved method and heartrate monitor system for alerting a user to his/her heartrate, thus contributing to the effectiveness of a workout, strenuous exercise, stress regimen or the like.

### BACKGROUND OF THE INVENTION

Physical exercise has been shown to be beneficial to an individual's mental and physical health. The direct benefits of aerobic exercise include muscle strength, flexibility, increased endurance and vigor. Additionally, exercise has been shown to assist in preventing disease and heart attacks, as well as lowering cholesterol and blood pressure, among other things.

During exercise, the idea of maintaining one's heartrate in a target zone (“Target Zone”), defined as some elevated range above where the person's heartrate is at normal rest, has been shown to be an important part of optimizing physical conditioning. For example, cardiovascular benefits may be achieved by exercising at an individual's target heartrate (“Target Heartrate”) for 30 minutes at least three times per week. The Target Heartrate is usually considered to be a percentage of an individual's maximum heartrate (MHR). A generally accepted methodology for approximating an individual's MHR is by subtracting the his/her age from 220. For an unconditioned individual, the target rate may be 60%-70% of the MHR; for well conditioned individuals, the target rate may reach 85% of the MHR. Clearly then, the Target Heartrate falls within the Target Zone.

Devices and methods for measuring an individual's heartrate are well known in the prior art. For example, U.S. Pat. No. 6,026,335 describes a chest belt apparatus and methodology for monitoring a user's heart rate within a defined target-zone during exercise. The user will input his/her age, and the device will compute the target heart rate based on the individual's age-based Maximal Heart Rate.

U.S. Pat. No. 6,345,197 to Fabrizio teaches a method and apparatus to monitor a user's heart rate within a defined target-zone. The user may program the device with information such as the upper and lower heart rate values defining the target-zone and the desired training time in target-zone. During exercise, the device will alert the user when he/she is outside the target-zone.

U.S. Pat. No. 4,566,461 to Lubell et al. even goes as far as recognizing that it may be desirous of changing the audible pitch of the alarm as the sampled heartrate decreases (i.e. lower pitch) or increases (higher pitch).

As the foregoing examples make clear, the prior art describes the ability to alert a user when the sampled heartrate is outside the Target Zone.

However, it is believed that the prior art methodologies are less than desirable in at least the following respects. For example, just before beginning his/her workout, a user may activate the heartrate monitoring feature. Expectedly, the device will then immediately alert the user that he/she is not in the Target Zone. The alert is silenced only after the user exercises sufficiently such that the sampled heartrate values reaches (and remain above) at least the lower value of the Target Zone (i.e. the “minimum threshold value”). Therefore, during the warm-up stage (i.e. until the sampled heartrate value reaches the minimum threshold value), the alarm may continuously and annoyingly indicate that the sampled heartrate has not yet reached the minimum threshold value, a fact that may be obviously recognized by the user without the aid of the alarm.

As but another example, upon completion of the workout, a user may typically “cool-down” by slowing the activity/exertion level, resulting in a corresponding decrease in the user's heartrate. With known prior art devices and methods, the user is thus forced to either endure unnecessary “out of zone” alarm indications during this “cool-down” since the device dutifully advises the user that he/she is outside the Target Zone, or interrupt the “cool-down” activity to manually disable the out of zone alert/alarm indicator.

Lastly, the prior art methods and devices are perceived to be less than desirable if operating during a somewhat out of the ordinary event within a larger workout (e.g. serial sprinting and cross training and/or extended rest period). For example, when the user wishes to engage in serial sprinting in an effort maintain a relatively faster target rate for a relatively short duration, the user will be forced to either discontinue the alarm indication after each sprint by manual deactivation or be distracted by the continual alert/alarm indication informing the user what she already knows, namely that he/she is temporarily above the upper limit of the Target Zone (i.e. the maximum threshold value).

The inventor of the present invention has found that alternative methodologies to overcome the aforementioned perceived deficiencies and further improve upon the state of the art and provide for yet unrealized advantages, are both desirable and achievable. A device and methodology that overcomes such deficiencies and improves the state of the art is embodied in the present invention disclosed herein. Specifically, the present invention overcomes perceived deficiencies in the prior art regarding undesirable and/or unnecessary alarm indications during, by way of example and not limitation, the warm up and cool-down phases of an exercise routine. However, the present invention is not limited thereby as another perceived deficiency which is overcome by the present invention is the ability to also avoid unnecessary or undesirable out of zone indications until a user's heartrate has slowed down to be below a predetermined maximum threshold as well.

### SUMMARY AND OBJECTS OF THE INVENTION

Therefore, it is an object of the present invention to overcome the perceived deficiencies in the prior art, such as by providing a novel method of monitoring a user's heartrate that avoids the continuous, unnecessary, and repetitive alert/alarm indications associated with prior art devices and methodologies.

Another object of the present invention is to provide an improved methodology used to alert users to their sampled heartrate during a workout, strenuous activity, stress regimen (e.g. one that requires the slowing down of one's heartrate) or other activity.

It is thus another object of the present invention to improve the exercise or workout experience and/or one's health regimen.

Therefore, generally speaking, the present invention is directed to a method of generating an out of zone alert when a sampled biomedical value does not satisfy a condition, wherein the generation of the out of zone alert is performed by an indicator, and the sampled biomedical value is determined in a biomedical value monitoring system, and further wherein the indicator does not generate the out of zone alert when the sampled biomedical value fails to satisfy the condition until a sampled biomedical value is determined to satisfy the condition, the method comprising the steps of determining when a sampled biomedical value satisfies the condition; and thereafter, permitting the generation of the out of zone alert when the sampled biomedical value is determined not to satisfy the condition.

In a particular embodiment, the invention is directed to a method of generating an out of zone alert when a sampled heartrate value is below a threshold minimum value, wherein the indicator does not generate the out of zone alert when the sampled heartrate value is below the threshold minimum value until the threshold minimum value has been reached, wherein the method comprises the steps of determining when a sampled heartrate value is at or above a threshold minimum value; and permitting the generation of the out of zone alert when the sampled heartrate value falls below the threshold minimum value.

In another particular embodiment, the invention is directed to a method of generating an out of zone alert when a sampled heartrate value is above a threshold maximum value, and wherein the indicator does not generate the out of zone alert when the sampled heartrate value is above the threshold maximum value until the threshold maximum value is greater than a sampled heartrate value, the method comprising the steps of determining when the threshold minimum value is greater than a sampled heartrate value; and permitting the generation of the out of zone alert when a subsequent sampled heartrate value is above the threshold maximum value.

In still yet a further embodiment, the method is directed to the generation an out of zone alert when a sampled heartrate value is outside a Target Zone, and comprises the steps of determining when a sampled heartrate is within the Target Zone; and thereafter permitting the generation of the out of zone alert when the sampled heartrate is determined to be outside the Target Zone.

Lastly, in yet another preferred embodiment, the present invention is directed to a method of generating an out of zone alert when a sampled heartrate value does not satisfy a condition, wherein the generation of the out of zone alert is performed by an indicator, and the sampled heartrate value is determined in a heartrate monitoring system, the method comprising the steps of determining whether the sampled heartrate fails to satisfy a condition for more than a continuous period of time, and if so, suppressing further generation of the out of zone alert even if a next successive sampled heartrate value fails to satisfy the condition, and if not, generating the out of zone alert. In a particular application, the condition may be whether the sampled heartrate value is outside a Target Zone, is above a threshold minimum value, and/or below a threshold maximum value.

### BRIEF DESCRIPTION OF THE DRAWINGS

For a fuller understanding of the invention, reference is made to the following accompanying drawing, in which:

FIG. 1 is a flowchart illustrating a methodology in accordance with the present invention.

### DESCRIPTION OF THE PREFERRED EMBODIMENT

Generally speaking, the present invention, as one skilled in the art will readily appreciate, can be incorporated into a wide array of heartrate monitor devices. That is, the present invention is in no way limited to one particular heartrate monitoring device or system, and is thus applicable to devices/systems that (i) compute sampled heartrate values in the cheststrap and then transmit the computed heartrate values to the accompanying wristworn device, (ii) compute the sampled heartrate values in the wristworn device watch only after data representative of the sampled heartrate is taken in the cheststrap and transmitted to the wristworn device, (iii) sample, compute and display the sampled heartrate value in the wristworn device or the cheststrap, or (ii) any variation or combination of the foregoing. Therefore, the disclosures of the aforementioned cited patents, namely U.S. Pat. Nos. 6,026,335; 6,345,197 and 4,566,461, to the extent they describe how a heartbeat is measured or sampled, how a heartrate value is computed and/or how information relating to the heartrate is displayed on a device (i.e. a piece of exercise equipment) and/or transmitted to another device, such as a wristworn device, and how such a heartrate is displayed or conveyed to a user, along with the design, construction and implementation of an alarm (audible and/or visible), are all incorporated by reference as if fully set forth herein. To ensure completeness, the disclosure of U.S. Pat. No. 6,332,094 is also incorporated by reference as if fully set forth herein for its similar descriptions.

Therefore, and notwithstanding the fact that one skilled in the art could, from the aforementioned disclosures which are incorporated by reference, adequately and easily design and construct a heartrate monitor that could utilize and implement the present invention which will hereinafter be disclosed, a few specific details of the preferred heartrate monitor construction is set forth.

Specifically, in the preferred construction, the methodology of the present invention is incorporated into a heartrate monitoring system comprising (i) a transmitter preferably located in a cheststrap and (ii) a wristworn unit. In this system, the transmitter transmits a packet of data, comprising among other things the computed sampled heartrate value, to the wristworn device, in predetermined time intervals, such as by way of example and not limitation, every two seconds. Thus, it is clear that the transmission rate may be independent of the heartrate that the transmitter is measuring. The receiver (i.e. the wristworn device) receives the packet of data, processes the information, and displays the heartrate values. Preferably, the wristworn device, exercise equipment or the like, generates an out of zone alert based on a signal(s) from a microprocessor (again, preferably located in the exercise requirement or the wristworn device, as the case may be), which processes the data received from the transmitter to determine whether the sampled heartrate value satisfies a given condition, such as being within or outside a set Target Zone. An audio amplifier and audio transducer may be provided to generate an audible out of zone indication, while a display may be provided to generate a visible out of zone indication. Processing of the signals to generate the out of zone alert/alarm is clearly within the scope of the ordinarily skilled artisan.

By way of background, the methodology of the present invention utilizes three values, namely the threshold minimum value which represents the lower end of the Target Zone, the threshold maximum value which represents the upper end of the Target Zone, and the “timeout value” which represents length of time the user wishes the alarm to be generated (audibly and/or visibly) after the sampled heartrate value has fallen below the threshold minimum value or has risen above the threshold maximum value. All three values may be individually imputed into the device or system or may be computed by the device or system. Both features are well known in the art.

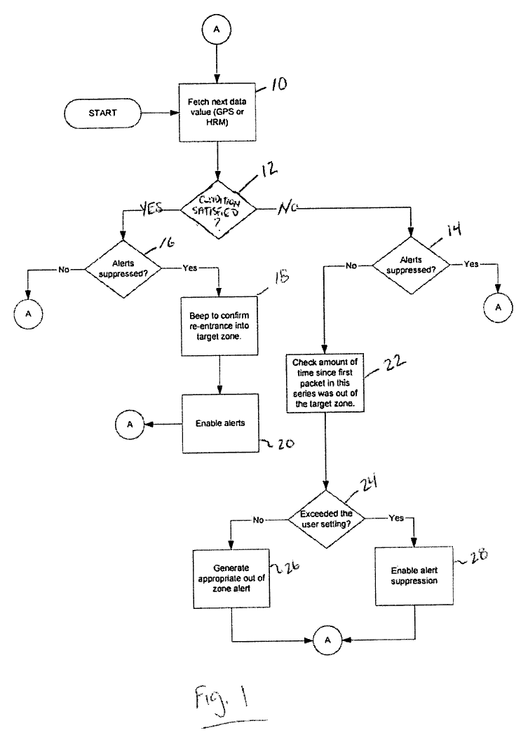

Furthermore, although not explicitly illustrated in the figures, it should be understood to a routine programmer that one or more flags may be used to keep track of conditions, as would be well known in the art. Here for example, at least one flag is used, its name for ease of description being referred to as the ALERTSUPPRESSION flag. Preferably, this flag is set before beginning the routine set forth in FIG. 1, but this is by way of example and not limitation. Such minor modifications and details of the present invention are well within the scope of the ordinarily skilled artisan.

Turning now to the particulars of the present invention, one will readily see that FIG. 1 illustrates a method of generating an out of zone alert when a sampled biomedical value does not satisfy a condition, wherein the generation of the out of zone alert is performed by an indicator, and the sampled biomedical value is determined in a biomedical value monitoring system, and further wherein the indicator does not generate the out of zone alert when the sampled biomedical value fails to satisfy the condition until a sampled biomedical value is determined to satisfy the condition. In this general embodiment, the method comprises the steps of determining when a sampled biomedical value satisfies the condition; and thereafter, permitting the generation of the out of zone alert when the sampled biomedical value is determined not to satisfy the condition.

In a particular application, as will be exemplified below, the method of generating an out of zone alert may be particularly applicable when a sampled heartrate value is below a threshold minimum value. Here, the indicator does not generate the out of zone alert when the sampled heartrate value is below the threshold minimum value until the threshold minimum value has been reached, and the method comprises the steps of determining when a sampled heartrate value is at or above a threshold minimum value; and permitting the generation of the out of zone alert when the sampled heartrate value falls below the threshold minimum value.

Specific features of this application may include the step of enabling the indicator so that the indicator will generate the out of zone alert when the sampled heartrate falls below the threshold minimum value, and/or including the steps of repeatedly sampling heartrate values until a sampled heartrate value is at or exceeds the minimum threshold value and maintaining the inability to generate out of zone alerts until a sampled heartrate value is determined to be equal to or greater than the threshold minimum value, and/or including the steps of determining a next sampled heartrate value and determining that it is less than the threshold minimum value and generating the out of zone alert. Advantageously, the generation of the out of zone alert may be performed audibly and/or visually. Also, the threshold minimum value may be manually inputted into and/or calculated by the heartrate monitoring system.

In another particular application, the method is directed to generating an out of zone alert when a sampled heartrate value is above a threshold maximum value. Similarly, here the indicator does not generate the out of zone alert when the sampled heartrate value is above the threshold maximum value until the threshold maximum value is greater than a sampled heartrate value. Specifically, the method comprises the steps of determining when the threshold minimum value is greater than a sampled heartrate value; and permitting the generation of the out of zone alert when a subsequent sampled heartrate value is above the threshold maximum value.

Similarly, there may be specific features customized for this application, such as including the steps of enabling the indicator so that the indicator will generate the out of zone alert when a sampled heartrate value exceeds the threshold maximum value; and/or repeatedly sampling heartrate values until the maximum threshold value exceeds a sampled heartrate value and maintaining the inability to generate out of zone alerts until the maximum threshold value exceeds a sampled heartrate value; and/or determining a next sampled heartrate value and determining that it is greater than the threshold maximum value and generating the out of zone alert based thereon. Likewise, the generation of the out of zone alert may be performed audibly and/or visually. Also, the threshold maximum value may be manually inputted or computed.

As should now be clear, the method may combine aspects of the foregoing and generate an out of zone alert when a sampled heartrate value is outside a Target Zone, as will be disclosed below.

At this point it is helpful to provide practical examples of the present invention:

In one application, a user may set the Target Zone at between 100 and 130, thereby defining the threshold minimum value as 100 and the threshold maximum value as 130. Further, for purposes of illustration, it is assumed that the user's resting heartrate is 60. When the user first begins exercising, and in accordance with a first aspect of the invention, the present invention will suppress indication that the user is out of (i.e. below) the Target Zone until the sampled heartrate is first at or above the threshold minimum value.

In yet another application, the user may set the Target Zone of between 60 and 70, thereby defining the threshold minimum value as 60 and the threshold maximum value as 70. For purposes of illustration, it is assumed that the user's heartrate begins at an elevated 130 (e.g. the user does not initiate the heartrate monitor until the user's heartrate is quite elevated). Here, and in accordance with this second aspect of the invention, the present invention will suppress indication that the user is out of the Target Zone until the sampled heartrate first dips to or below the threshold maximum value.

As can thus be seen in the first application, there is no generation of an out of zone alert to indicate that the sampled heartrate is below the threshold minimum value until the threshold minimum value has been reached for a first time, while in the second application the indicator does not indicate that the sampled heartrate is above the threshold maximum value until the threshold maximum value has been passed (on the decreasing side) for the first time. Methodologies for all of the foregoing applications will be exemplified by a detailed consideration of FIG. 1.

For example, once in the routine of FIG. 1, control passes to Step 10 wherein the receiver (e.g. a piece of exercise equipment (e.g. treadmill or stationary bike) or a wristworn device) receives a computed sample heartrate value from the transmitter (i.e. located in a cheststrap or possibly in the device itself (i.e. in a grip of a piece of equipment or in the wristworn device). However, to ensure completeness and properly recognize the broad scope of the present invention, it should be understood that this step could easily be replaced or supplemented by a step that provides that the receiver (also) receives data representative of the sampled heartrate value and wherein the computation of the sampled heartrate value occurs in the device that incorporates the receiver (i.e. the equipment or the wristworn device). Variations thereof are all included within the scope of the present invention, although they are not material thereto.

Control then passes to Step 12 wherein it is determined whether the sampled heartrate value satisfies the predetermined condition, which in the preferred embodiments may be whether the heartrate value is within the Target Zone, above a minimum threshold value and/or below a maximum threshold value. Keeping in mind an objective of the present invention, namely to generate an out of zone alert only after the sampled heartrate has satisfied the condition, control of the routine then passes to Step 14 if the sampled heartrate did not satisfy the condition.

Step 14 in effect determines whether the sampled heartrate has satisfied the condition at least once and/or whether the condition has not been satisfied for such an extended period (i.e. the “time-out” period) that satisfaction of the condition again is required prior to the generation of the out of zone indication. The time-out period is very much user specific and based clearly on user desired parameters, although the manufacturer may also set such a time-out period. For example, 15 or 30 seconds appear to be desirable, although this is by way of example and not limitation.

For example, if the answer to the query at Step 14 is “yes”, control again returns to point “A” since it has been determined that either (i) at no prior point has the sampled heartrate satisfied the condition or (ii) the condition has not been satisfied for such an extended period (i.e. the time-out period) that satisfaction of the condition is again required prior to the generation of the out of zone indication. Therefore, in keeping with the objectives of the present invention, no alert/alarm is generated.

On the other hand, if the answer at Step 14 is “no”, then it can be assumed that the sampled heartrate has previously satisfied the condition and the sampled biomedical (e.g. heartrate) value has not continuously failed to satisfy the condition for more than the “time-out” period required to disable the alarm (Step 24 discussed further below). That is, at Step 14 the status of the ALERTSUPPRESSION flag is determined.

To better appreciate the “no” answer to the inquiry at Step 14, it is important to first return to Step 12, recalling this gatekeeper decision inquiry as to whether the sampled heartrate (obtained at Step 10) satisfied the condition (i.e. within the Target Zone, above the minimum threshold value and/or below the maximum threshold value). Hence, a “yes” to the inquiry at Step 12 is an indication that the sampled heartrate value did satisfy the condition (e.g. within the Target Zone, above the minimum threshold value or below the maximum threshold value), and control would pass to Step 16 where it is determined whether the ALERTSUPPRESSION flag is still set. If it is (thus implying that the previous sampled heartrate value failed to satisfy the condition), control passes to Step 18 where a confirmation “beep” may be made to confirm/indicate that the sampled heartrate has now satisfied the condition (e.g. within the Target Zone). Recalling that the ALERTSUPPRESSION flag remains set until the sampled heartrate value satisfies the condition of Step 12, the ALERTSUPPRESSION flag is then cleared at Step 20 so that any subsequent sampled heartrate values determined at Step 12 to satisfy the condition do not receive any further confirmation “beeps” (i.e. at Step 20) since all further inquiries at Step 16 as to whether the ALERTSUPPRESSION flag is set will be answered in the negative, and hence no beeping will occur and control will again pass back to point “A”.

Therefore, continuing on with the methodology subsequent to the determination at Step 14 that the ALERTSUPPRESSION flag has been cleared (i.e. at Step 20 as a result of a previous sampled heartrate value satisfying the condition at Step 12), control would then pass to Step 22 to determine, compute or otherwise calculate the amount of time that has continuously elapsed since the last sampled heartrate value met the condition at Step 12.

If the condition at Step 12 has not been satisfied for less than the “time-out” value (determined at Step 24), then control passes to Step 26 where the indicator generates the out of zone alert. Control then passes back to point “A” in anticipation of another sampled heartrate value (or data related thereto). On the other hand, timing out of the timer/counter at Step 22 indicates that no consecutive sampled heartrate value has satisfied the condition at Step 12 for more than the predetermined “time-out” period, and the generation of the out of zone indicator will be suppressed (Step 28) (i.e. ALERTSUPPRESSION flag is again set). In this way, all further successive sampled heartrates values that fail to satisfy the condition set in Step 12 will cause the routine to follow the sequence of Steps 12 and 14, culminating with the query at Step 14 being answered in the positive, hence returning to point “A” without generating the out of zone alert. Clearly, once the condition is satisfied for a subsequent sampled heartrate, the timer/counter must be reset, such as would clearly be understood by one ordinarily skilled in the art.

As such, it can be seen that this last aspect of the present invention is directed to a method of generating an out of zone alert when a sampled heartrate value does not satisfy a condition, wherein the generation of the out of zone alert is performed by an indicator, and the sampled heartrate value is determined in a heartrate monitoring system, the method comprising the steps of determining whether the sampled heartrate fails to satisfy a condition for more than a continuous period of time, and if so, suppressing further generation of the out of zone alert even if a next successive sampled heartrate value fails to satisfy the condition, and if not, generating the out of zone alert. Exemplary conditions are whether the sampled heartrate value is within a Target Zone, merely above a threshold minimum value or below a threshold maximum value.

Hence, it can be seen that the foregoing methodology provides for an improved and more pleasurable workout or exercise experience, since, once it is assumed that the exercise/workout objective is to get one's heartrate into a Target Zone, above the minimum threshold value or below the maximum threshold value, the present invention overcomes the perceived annoyances and/or deficiencies in the prior art by suppressing any indications until the user's heartrate has entered and/or reached the desired thresholds.

A simple example highlights the advantageous nature of the invention:

A jogger wishes to engage in a long jog at a desired heart rate of between 110 and 140; if he rests or sprints, he desires that the out of zone alarm does not sound for more than 30 seconds. The user's goal is to reenter the target-zone before the 30-second alarm is silenced automatically. The jogger inputs the minimum, maximum, and timeout values. With a resting heart rate of 50, the user begins to jog. After 15 seconds, his heart rate reaches 110, and the device is armed. After 30 seconds, he reaches a heart rate of 120. The user's hear rate remains at 120 for one minute and then he stops running and rests. After 45 seconds, his heart rate drops below 110, and the alert sounds. The user wishing to maintain his target heart rate, rests for the first 15 seconds of the alert, and starts jogging again. After 10 seconds, his heart rate reaches 110, the target-zone is reentered, the device signals the reentry and the alarm is silenced because the target-zone has been reentered after 25 seconds. Had the runner failed to reach 110 after 30 seconds, the alert would have been silenced because the timeout value was reached.

Hence, it can be seen that the foregoing methodology provides for an improved and more pleasurable workout or exercise experience, since, once it is assumed that the exercise/workout objective is to get one's heartrate into a Target Zone, the present invention overcomes the perceived annoyances and/or deficiencies in the prior art by suppressing any indications until the user's heartrate has entered and/or reached the desired thresholds.

It will thus be seen that the objects set forth above, among those made apparent from the preceding description, are efficiently attained and, since certain changes may be made in the above methodologies without departing from the spirit and scope of the invention, it is intended that all matter contained in the above description or shown in the accompanying drawing shall be interpreted as illustrative and not in a limiting sense.

Finally, it should be well appreciated that the present invention is well suited for a wide variety of electronic biomedical devices, only one of which is a heartrate monitor. Therefore, while the present disclosure provides its preferred embodiment as being a heartrate monitor, the invention should not be deemed to be so limited. That is, it should be clear that any electronic device that could utilize the present invention is intended to be covered hereby.

1. A method of generating an out of zone alert when a sampled biomedical value does not satisfy a condition, wherein the generation of the out of zone alert is performed by an indicator, and the sampled biomedical value is determined in a heartrate monitoring system,
the method comprising the steps of:
not generating the out of zone alert when the sampled biomedical value fails to satisty the condition until a sampled biomedical value is determined to satisfy the condition;
determining when the a sampled biomedical value first satisfies the condition; and
thereafter, permitting the generation of the out of zone alert when the sampled biomedical value is determined not to satisfy the condition.

2. A method of generating an out of zone alert when a sampled heartrate value is below a threshold minimum value, wherein the generation of the out of zone alert is performed by an indicator, and the sampled heartrate value is determined in a heartrate monitoring system,
the method comprising the steps of:
not generating the out of zone alert when the sampled heanate value is below the threshold minimum value until the threshold minimum value has been reached;
determining when the sampled heartrate value is at or above the a threshold minimum value; and
permitting the generation of the out of zone alert when the sampled heartrate value falls below the threshold minimum value.

3. The method as claimed in claim 2, including the step of enabling the indicator so that the indicator will generate the out of zone alert when the sampled heartrate falls below the threshold minimum value.

4. (canceled)

5. The method as claimed in claim 2, including the steps of:
determining a next sampled heartrate value and determining that it is less than the threshold minimum value; and
generating the out of zone alert.

6. The method as claimed in claim 5, wherein the generation of the out of zone alert is performed audibly and/or visually.

7. The method as claimed in claim 2, including the step of inputting the threshold minimum value into the heartrate monitoring system.

8. A method of generating an out of zone alert when a sampled heartrate value is above a threshold maximum value, wherein the generation of the out of zone alert is performed by an indicator, and the sampled heartrate value is determined in a heartrate monitoring system,
the method comprising the steps of:
not generate not generating the out of zone alert when the sampled heartrate value is below the threshold minimum value until the threshold minimum value has been reach;
determining when the sampled heartrate value is above the threshold maximum value; and
permitting the generation of the out of zone alert when a subsequent sampled heartrate value is above the threshold maximum value.

9. The method as claimed in claim 8, including the step of enabling the indicator so that the indicator will generate the out of zone alert when a sampled heartrate value exceeds the threshold nmaxiimum value.

10. The method as claimed in claim 8, including the steps of:
repeatedly sampling heartate values until the maximum threshold value exceeds a sampled heartrate value; and
maintaining the inability to generate out of zone alerts until the maximum threshold value exceeds a sampled heartrate value.

11. The method as claimed in claim 8, including the steps of:
determining a next sampled heartrate value and determining that it is greater than the threshold maximum value; and
generating the out of zone alert

12. The method as claimed in claim 8, wherein the generation of the out of zone alert is performed audibly and/or visually.

13. The method as claimed in claim 8, including the step of inputting the threshold maximum value into the heartrate monitoring system.

14. A method of generating an out of zone alert when a sampled heartrate value is outside a Target Zone, wherein the generation of the out of zone alert is performed by an indicator, and the sampled heartrate value is determined in a heartrate monitoring system,
the method comrprsing the steps of:
not generating the out of zone alert when the sampled heartrate value is below the threshold minimum value until the threshold minimum value has been reached;
determining when a sampled heartrate is first within the Target Zone; and
thereafter permitting the generation of the out of zone alert when the sampled heartrate is determined to be outside the Target Zone.

15. A method of generating an out of zone alert when a sampled heartrate value does not satisfy a condition, wherein the generation of the out of zone alert is performed by an indicator, and the sampled heartrate value is determined in a heartrate monitoring system, the method comprising the steps of:
determining whether the sampled heartrate fails to satisfy a th condition for more than a predetermined continuous period of time, and if so:
suppressing furter generation of the out of zone alert even if a next successive sampled heartrate value fails to satisfy the condition, and continually suppressing further generation of the out of zone alert for successive sampled heartrate values that fail to satisfy the condition until a sampled heartrate value satisfies the condition, and when the sampled heartrate value does satisfy the condition, indicating that the sampled heartrate value has satisfied the conditon, if not:
generating the out of zone alert.

16-17. (canceled)

18. The method as claimed in claim 15, including the step of generating an out of zone indication if a successive sampled heartrate value fails to satisfy the condition.

19. The method as claimed in claim 15, wherein the condition is that the sampled heartrate value is within a Target Zone.

20. The method as claimed in claim 15, wherein the condition is that the sampled beartrate value is above a threshold minimum value.

21. The method as claimed in claim 15, wherein the condition is that the sampled heartrate value is below a threshold maximum value.
